(19) United States
(12) Patent Application Publication
(10) Pub. No.: US 2007/0259428 A1
(43) Pub. Date: Nov. 8, 2007

(54) Use of Cloud Point System in Biotransformation

(75) Inventors: Daijie Chen, Shanghai (CN); Zhilong Wang, Shanghai (CN); Mei Ge, Shanghai (CN); Yiping Jin, Shanghai (CN); Weidong Ye, Shanghai (CN)

Correspondence Address: VENABLE LLP, P.O. BOX 34385, WASHINGTON, DC, 20043-9998, US

(73) Assignees: Shanghai Health Creation Center for Biopharmaceutical R&D Co., LTD., Shanghai (CN); Zhejiang Medicine Co., LTD. Xinchang Pharmaceutical Factory, Zhejiang (CN)

(21) Appl. No.: 10/567,439

(22) PCT Filed: Jul. 5, 2004
(86) PCT No.: PCT/CN04/00746

§ 371 Date: Mar. 14, 2007

(30) Foreign Application Priority Data
Aug. 7, 2003 (CN) 03142114.8

(51) Int. Cl. C12N 15/87

(52) U.S. Cl. 435/440

(57) ABSTRACT

The invention relates to the field of microbial technology. It discloses a method to apply the cloud point system (CPS) in biotransformation by selecting one or more types of nonionic surfactant to form a aqueous system with a cloud point below the microbial transformation temperature, which serves as transformation medium. The method disclosed is suitable in particular for microbial transformation of hydrophobic compounds, for the system where substrate or product inhibits microbial growth or where product is further degraded by microbes. The CPS in the present invention forms a microemulsion of water-in-oil and oil-in-water, where the drops of surfactant is able to solubilize, serving as substrate reservoir and product extractant. This enhances bioavailability of substrates and elimination of product inhibition. The large water vesicles existing in the continuous surfactant phase provide aqueous environment to the cells where they can be sheltered from detrimental effects of surfactants, resulting in improvement of biocompatibililty.

### FIELD OF THE INVENTION

The invention relates to the field of microbial technology, more specifically to application of cloud point system in biotransformation.

### BACKGROUND OF THE INVENTION

Biotransformation of hydrophobic compounds in aqueous medium are often hindered by some obstacles: a limited substrate accessibility to microbes as a result of the low aqueous solubility of most organics, inhibition or toxicity of both substrate and product exerted upon the microbes. Such problem commonly exists as well in the biodegradation process of toxic pollutants. Medium engineering is an attempt to alleviate or overcome these problems by adding different kinds of inherently biocompatible and non-biodegradable ingredients into the essentially aqueous medium to form various types of microbial transformation medium. Many medium systems such as aqueous organic two-phase system, aqueous two-phase polymer system, liposome medium, direct micelle system, water-in-oil microemulsion or reverse micelle system, have been reported.

When an aqueous micelle solution of a nonionic surfactant is at a temperature above its cloud point (CP) or in the presence of certain inducers, phase separation occurs to form a surfactant diluted phase and a surfactant-rich phase (coacervate phase), which is called cloud point system (CPS) and has been used in separation technology for years. The system is attractive because it provides a separation method which is easy to manipulate, reliable in scaling-up, simple in operation. Especially, it provides a watery and mild environment so that cells or proteins will be protected from damage. CPS should possess good biocompatibility, while both substrate and product would be able to partition in different phases according to properties of the phase system and the partitioned substance. In microbial transformation in a two-phase-based CPS, the potential toxicity or inhibition effect from substrate or product may be reduced. Moreover, CPS offers the possibility for replacing the conventional mechanical separation of cells from products with cell extraction process. Surfactants are known to increase apparent aqueous solubility of hydrophobic compounds (known as solubilization) and may be used to enhance the bioavailability and stimulate microbial transformation. Despite such many advantages mentioned above, CPS which serves as transformation medium has not yet be reported so far.

### DESCRIPTION

The present invention is to provide new application of cloud point system (CPS). It provides new application of CPS in biotransformation, by applying one or more types of nonionic surfactant to form a aqueous system with a cloud point below the microbial transformation temperature (i.e. CPS), which serves as transformation medium. Nonionic surfactants cited herein are selected from the group comprising polyoxyethylene alcohols, polyoxyethylene sorbitan fatty acid esters and alkylphenol ethoxylates. In particular, the polyoxyethylene alcohols comprise Brij 30, Brij 35, Brij 56 and C₁₂E₇; the polyoxyethylene sorbitan fatty acid esters comprise Tween 20, Tween 40, Tween 60, Tween 80, Span 20, Span 40, Span 60 and Span 80; the alkylphenol ethoxylates comprise Triton X-100 and Triton X-114.

The CPS disclosed in present invention is suitable in particular for:

1. Microbial transformation of hydrophobic compounds;

2. Where substrate or product inhibits microbial growth;

3. Where product would be further degraded by microbes;

Examples of its application includes side chain cleavage of cholesterol, transformation of steroids,
degradation of organic pollutants in sediment form, etc.

The present invention further describes selection of proper CPS as new method in medium engineering, which is based on the cholesterol side chain cleavage model.

An important intermediate, namely ADD, is formed following microbial cleavage of cholesterol side chain. This gives a typical example of biotransformation of hydrophobic compounds. Firstly, cholesterol is typically hydrophobic with solubility below 1 μM in water, while with other steroids it commonly ranges from 0.01 to 0.1 mM. Secondly, substrate as well as product in the system are toxic to the microbes.

Designing of the transformation system comprises:

1. Selection of Surfactants

Fourteen nonionic surfactants, which belong to three major classes, were chosen as candidates for screening. They are polyoxyethylene alcohols (Brij 30, Brij 35, from Fluka; Brij 56, C₁₂E₇, from Shanghai Surfactant Factory), polyoxyethylene sorbitan fatty acid esters (Tween 20, Tween 40, Tween 60, Tween 80, Span 20, Span 40, Span 60 and Span 80, all from Shanghai Reagent Co. Ltd.), alkylphenol ethoxylates (Triton X-100, from Shanghai Reagent Co. Ltd.; Triton X-114, from Fluka). Their basic properties are listed in Table 1.

TABLE 1
Basic properties of nonionic surfactants

| | | | | | |
|---|---|---|---|---|---|
| Nonionic | General | Hydrophobic | CMC | | CP |
| surfactant | structure* | group | (mM) | HLB | (° C.) |

Polyoxyethyene Alcohols

| | | | | | |
|---|---|---|---|---|---|
| Brij 30 | C₁₂E₄ | dodecanol | 0.02-0.06 | 9.5 | 4 |
| | C₁₂E₇ | dodecanol | 0.07 | 12.5 | 65 |
| Brij 35 | C₁₂E₂₃ | dodecanol | 0.09 | 16.9 | >100 |
| Brij 56 | C₁₆E₁₀ | blubber | | 12.9 | 64-69 |

Polyoxythyene Sorbitan Fatty Acid Esters

| | | | | |
|---|---|---|---|---|
| Span 20 | C₁₂S₆ | Lauric acid | | 8.6 |
| Span 40 | C₁₆S₆ | Palmitic acid | | 6.7 |
| Span 60 | C₁₈S₆ | Stearic acid | | 4.7 |
| Span 80 | C₁₈S₆ | Oleic acid | | 4.3 |
| Tween 20 | C₁₂S₆E₂₀ | Lauric acid | 0.04-0.06 | 16.7 |
| Tween 40 | C₁₆S₆E₂₀ | Palmitic acid | 29^{a} | 15.6 |
| Tween 60 | C₁₈S₆E₂₀ | Stearic acid | 27^{a} | 14.9 |
| Tween 80 | C₁₈S₆E₂₀ | Oleic acid | 0.01-0.02 | 15 |

Alkyphenol Ethoxylate

| | | | | | |
|---|---|---|---|---|---|
| Triton X-100 | C₈ΦE₉₋₁₀ | octylphenol | 0.2 | 13.5 | 64 |
| Triton X-114 | C₈ΦE₇₋₈ | octylphenol | 0.3 | 12.8 | 22 |

*S₆, sorbitan ring; Eₙ, the number of ethylene oxide group; Cₙ, the number of carbons in the alkyl chain; Φ, phenolic ring.
^{a}mg/L.

2. Microbial Transformation

Microbial strain, Mycobacterium sp. NRRL B 3683, is preserved in Shanghai Health Creation Center for Biopharmaceuticals R&D. It is able to remove the side chain of cholesterol, giving out ADD and 4-AD as final products, at a ratio of ca. 10:1.

Media

Slant culture medium (100 ml): yeast extract 0.5 g, agar 1.2 g, glycerol 1.0 g, H₂KPO₄ 0.05 g, (NH₄)SO₄ 0.1 g, MgSO₄.7H₂O 0.05 g

Seed culture medium (100 ml): (NH₄)SO₄ 0.5 g, Na₂HPO₄ 0.45 g, KH₂PO₄ 0.34 g, MgSO₄.7H₂O 0.05 g, glycerol 1.0 g, cholesterol 0.2 g , Triton X-100 0.2 g

Transformation medium (100 ml): (NH₄)SO₄ 1.0 g, Na₂HPO₄ 0.45 g, KH₂PO₄ 0.34 g, MgSO₄.7H₂O 0.2 g, cholesterol 0.5 g, nonionic surfactant 2.0 g

Microbial Cultivation

The seed culture was grown aerobically at 28° C. at 220 r/min for 3 days with 20 ml of medium in a 250 ml Erlenmeyer flask. The seed culture was then transferred by 10% into 22 ml of transformation medium in a 250 ml Erlenmeyer flask, which was then shaken at 28° C. at 220 r/min for 7 days. A portion of the well-mixed transformation culture broth was withdrawn for analysis.

Analysis of the outcomes

1. HPLC

1 ml of sample was withdrawn from culture broth and extracted with 4 ml of methanol for 2 hrs, followed by centrifugation. 0.8 ml of supernatant was taken for HPLC analysis. Hypesil C18 column using methanol:water (4:1) as mobile phase, flow rate of 0.7 ml/min, and detection wavelength at 254 nm were applied. Profile for 4-AD and ADD is shown in FIG. 1.

2. TLC

TLC analysis was performed to determine the distribution of substrate and products in CPS, using Silica gel 60 F₂₅₄ (Merck) high-performance thin layer chromatography plates which is developed in Chloroform:ether (1:3) followed by soaking in phosphornolybdic acid solution and drying by heat. Substrate and products were separated, with their R_{f} values of cholesterol, a-AD and ADD being 0.62, 0.47, and 0.42, respectively.

3. Determination of Cloud Point

Temperature at which solution turned cloudy was checked by visual observation. Temperature was raised in small increments, and the turning point was recorded when the solution became cloudy. Then the temperature was lowered slowly until the cloud disappeared. Record this second point. The mean value of the two temperature points mention above was taken as cloud point.

4. determination of Solubilization

Supersaturated ADD solution was prepared in a series of 2% surfactant solution, with different ratio between Triton X-100 and Triton X-114. Vials containing the solution were shaken at 220 r/min and 28° C. for 72 hrs, followed by filtration with a filter (20 μm pore-size, manufactured by Shanghai Institute of Pharmaceutical Industry) and HPLC analysis for ADD quantification.

Results and Evaluation

(1) Screening of Nonionic Surfactant

For selecting appropriate medium of microbial transformation, biocompatibility or potential toxicity of surfactant to microorganism is critical. Potential toxicity can be demonstrated by measuring the final product (ADD) concentration in surfactant-amended transformation medium. FIG. 2 shows the transformation result with different surfactant solutions.

At high concentration of surfactants, Triton X-114 was the only one which was able to form CPS, resulting in a maximum final amount of ADD. Triton X-100, which is in the same class with Triton X-114, was incompatible with the microorganism, indicating that CPS was responsible for the improvement of biocompatibility.

(2) Mechanisms for Improvement of Biocompatibility and Bioavailability with CPS

After phase separation of CPS, followed by staining with oil soluble dye Sudan black B, the medium system was observed under microscope. FIG. 3 shows the microscopy of dilute phase and coacervate phase of the said CPS. In the dilute phase, small surfactant drops or micelles were visualized as dark spots resulting from the formation of oil-in-water microemulsion. While in the coacervate phase, water-in-oil microemulsion was formed. The dark background shows the continuous surfactant phase, which acted as a substrate reservoir and product extractant. The large water vesicles existing in the continuous surfactant phase provided aqueous environment to the cells where they could be sheltered from detrimental effects of surfactants. Thus the biocompatibility of cells and surfactants was improved. In water vesicles inside the coacervate phase there exists oil-in-water microemulsion which is similar to that in the dilute phase. Mass transfer may occur at interface such as the site between surfactant drops, between vesicles, and between continuous phase and noncotinuous phase, whereas coalescence of the drops further enhances the rate of mass transfer. As a result, biocompatibility of hydrophobic compounds which are poor in water solubility is improved.

By TLC analysis, the partition of substrate and product in CPS is viewed in FIG. 4. Substrate solubility is greatly enhanced in the coacervate phase due to its favorable solubilization property. Similar to substrate, ADD is based in the coacervate phase as well. Partition of cells between the two phases is determined with hemocytometry. Its partition coefficient between the coacervate phase and the dilute phase is roughly 10, indicating the surface of Mycobacterium sp. is rather hydrophobic. This is in compliance with the report that Mycobacterium is a hydrophobic microbe.

(3) Solubilizing Capacity of the Enhanced CPS

Cloud point of the mixed system: As illustrated in FIG. 2, only the aqueous micelle solution based on Triton X-114 gives a cloud point, which is below the microbial transformation temperature, and forms a two-phase system, resulting in good biocompatibility. The cloud point and extent of solubilization of surfactant solution can be adjusted by adding different surfactant at a certain ratio to form a mixed surfactant micelle solution. To enhance the solubilizing capacity of CPS, Triton X-100 was chosen to form such a surfactant micelle solution. The cloud point of this mixed system is shown in FIG. 5. When the portion of Triton X-100 is over 20% by weight, the cloud point of the system reaches a level beyond the microbial transformation temperature of 28° C. Major components of the transformation medium affect the cloud point very slightly. ADD, as microbial transformation product, reduces the cloud point of the mixed system apparently. This may cause a cloud point below the cultivation temperature, resulting in the change from one-phase to two-phase.

Solubilization of the mixed system: The solubilization of the mixed surfactant micelle solution upon ADD varies with the portion of Triton X-114, as shown in FIG. 6. As single surfactant, Triton X-100 is superior to Triton X-114 in solubilization. However, in a mixed system where Triton X-114 is below 50%, the solubilizing capacity of the system decreases while Triton X-114 level drops to lower. When Triton X-114 reaches 70%, solubilization upon ADD becomes maximal. Referring to FIG. 3, surfactant in the coacervate phase exists in a totally different form from that in the micelle solution. This indicates solubilization of surfactant micelle solution is greatly affected by cloud point. FIG. 5 shows that ADD as product shares a same pattern of partition with the substrate in both phases in the CPS. From the data of ADD, it is proposed that the substrate has a similar solubilization property.

Microbial transformation in the mixed system: As result of a seven-days microbial transformation in a mixed system with different Triton X-114 levels, phase separation and product rate of ADD is shown in FIG. 7. It is indicated that phase separation occurs only when Triton X-100 is below 50%. In this case final concentration of ADD product is higher than that in the pure Triton X-100 system, as well as that in the pure Triton X-114 system. Its concentration varies with the change of Triton X-114 level, in a similar pattern as that in the solubilization upon ADD (FIG. 6), reaching a maximum when Triton X-114 level is 70%. A high ADD productivity can be attributed to the solubilizing behavior of the mixed system, which enhances dissolution of substrates and improves biocompatibility. In the mean time, inhibition from product is removed when it is extracted away.

The present invention achieves the microbial transformation in a CPS formed with Triton X-100 and Triton X-114, by side chain cleavage of cholesterol to get a product of ADD, which is one of the important intermediates of steroids. In this system, a microemulsion of water-in-oil and oil-in-water is generated. Drops of surfactant is able to solubilize, serving as substrate reservoir and product extractant. This is favorable to the substrate bioavailability, and to eliminate inhibition from product. The large water vesicles existing in the continuous surfactant phase provided aqueous environment to the cells where they could be sheltered from detrimental effects of surfactants. In addition, solubilization of the CPS can be adjusted by mixed surfactants. In a word, CPS is a promising approach in the field of medium engineering.

### DESCRIPTION OF THE DRAWINGS

FIG. 1 shows HPLC profiles of ADD and 4-AD

FIG. 2 shows ADD in transformation media with different surfactants

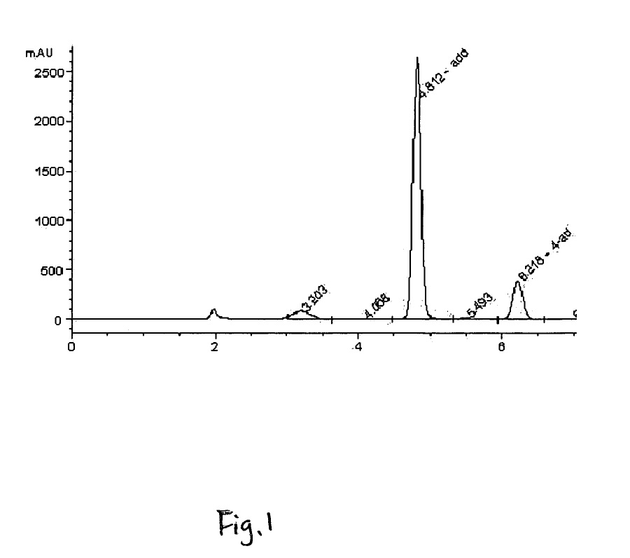

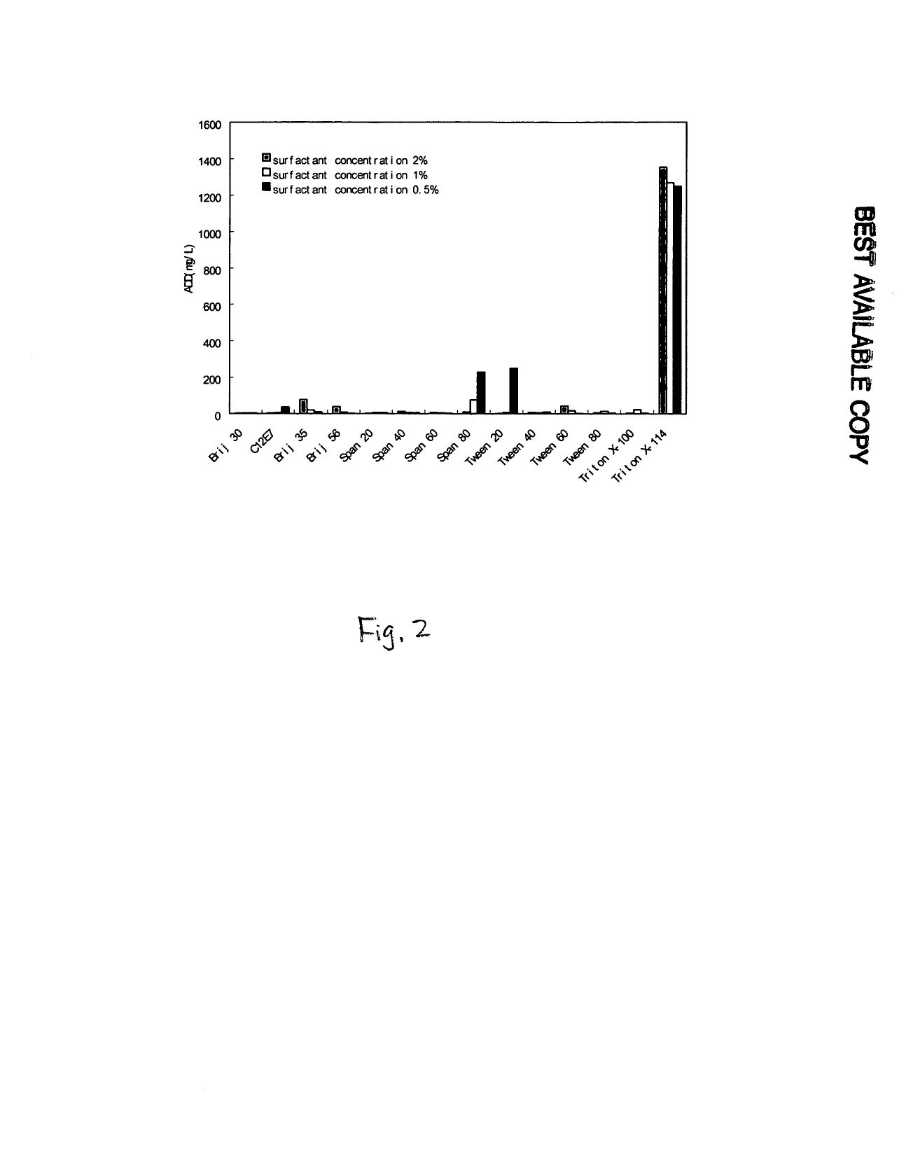

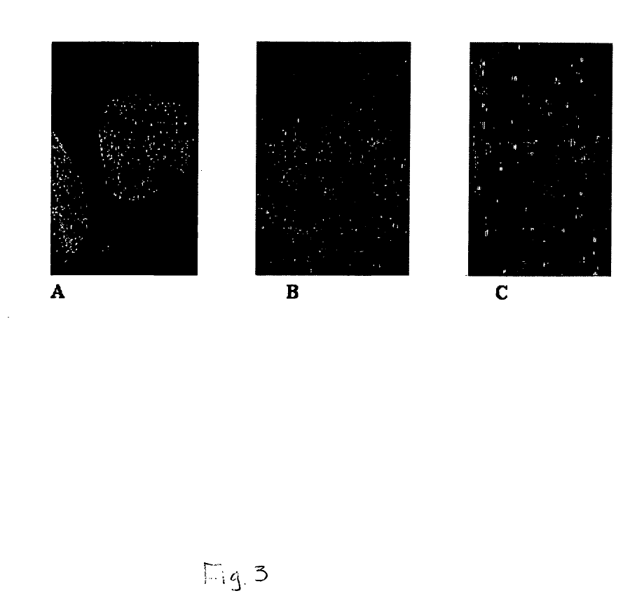

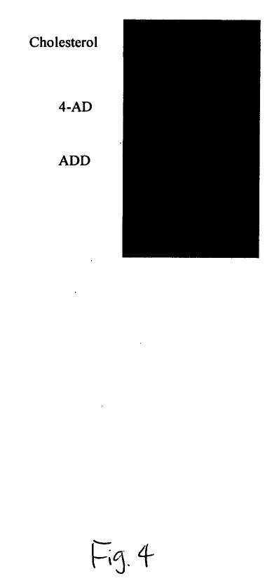

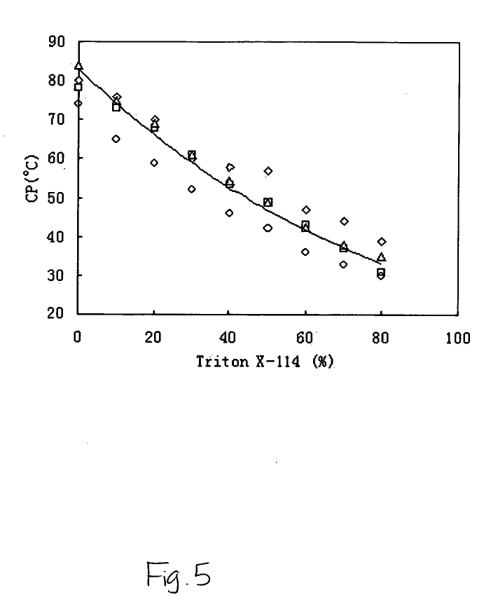

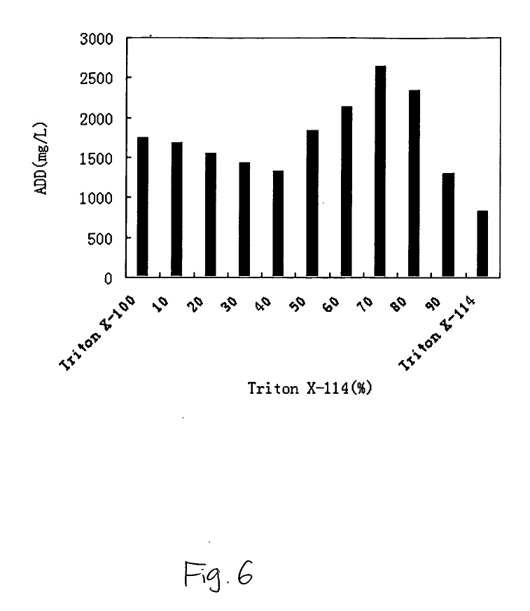

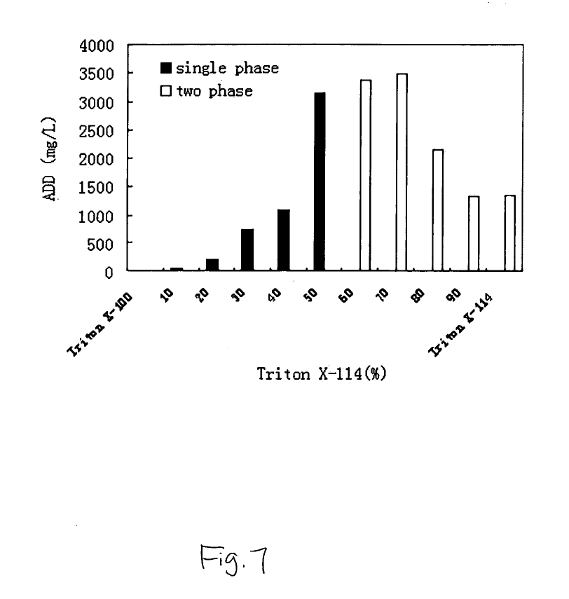

FIG. 3 shows Microscopic observation of dilute phase and coacervate phase
A: coacervate phase, water-in-oil emulsion (40×)
B: large water vesicle in coacervate phase, oil-in-water emulsion (600×)
C: dilute phase, oil-in-water emulsion (600×)

FIG. 4 shows Distribution of substrate and product in CPS
1. standards
2. in dilute phase
3. in coacervate phase

FIG. 5 shows Cloud point in mixed system
2.0 g nonionic surfactant in 100 ml of water (□); 1.0 g (NH₄)₂SO₄, 0.45 g Na₂HPO₄, 0.34 g KH₂PO₄, 0.2 g MgSO₄.7H₂O, 2.0 g nonionic surfactant in 100 ml of water (⋄); 1.0 g(NH₄)₂SO₄, 0.45 g Na₂HPO₄, 0.34 g KH₂PO₄, 0.2 g MgSO₄.7H₂O, 0.1 g cholesterol, 2.0 g nonionic surfactant in 100 ml of water (Δ); 1.0 g (NH₄)₂SO₄, 0.45 g Na₂HPO₄, 0.34 g KH₂PO₄, 0.2 g MgSO₄.7H₂O, 0.1 g ADD, 2.0 g nonionic surfactant in 100 ml of water (◯).

FIG. 6 shows Solubilization of mixed surfactant system.

FIG. 7 shows Phase separation and product level change with the fraction ratio of surfactant in the mixed system

### DESCRIPTION OF PREFERRED EMBODIMENTS

### EXAMPLE 1

Microbial strain Mycobacterium sp. NRRL B 3683 is able to remove the side chain of cholesterol, giving out ADD and 4-AD as final products, at a ratio of ca. 10:1. Slant culture medium (100 ml): yeast extract 0.5 g, agar 1.2 g, glycerol 1.0 g, H₂KPO₄ 0.05 g, (NH₄)SO₄ 0.1 g, MgSO₄.7H₂O 0.05 g

Seed culture medium (100 ml): (NH₄)SO₄ 0.5 g, Na₂HPO₄ 0.45 g, KH₂PO₄ 0.34 g, MgSO₄.7H₂O 0.05 g, glycerol 1.0 g, cholesterol 0.2 g, Triton X-100 0.2 g

Transformation medium (100 ml): (NH₄)SO₄ 1.0 g, Na₂HPO₄ 0.45 g, KH₂PO₄ 0.34 g, MgSO₄.7H₂O 0.2 g, cholesterol 1.45 g, mixture of Triton X-100 and Triton X-114 (1:1) 10.0 g

Microbial Cultivation

The seed culture was grown aerobically at 28° C. at 220 r/min for 3 days with 20 ml of medium in a 250 ml Erlenmeyer flask. The seed culture was then transferred by 10% into 22 ml of transformation medium in a 250 ml Erlenmeyer flask, which was then shaken at 28° C. at 220 r/min for 7 days. A portion of the well-mixed transformation culture broth was withdrawn for analysis.

Result

Total concentration of ADD and 4-AD (10:1) was 10 g/L, at a molar transformation rate of 93%.

### EXAMPLE 2

Microbial strain Mycobacterium sp. NRRL B 3683 is able to remove the side chain of cholesterol, giving out ADD and 4-AD as final products, at a ratio of ca. 10:1.

Slant culture medium (100 ml): yeast extract 0.5 g, agar 1.2 g, glycerol 1.0 g, H₂KPO₄ 0.05 g, (NH₄)SO₄ 0.1 g, MgSO₄.7H₂O 0.05 g

Seed culture medium (100 ml): (NH₄)SO₄ 0.5 g, Na₂HPO₄ 0.45 g, KH₂PO₄ 0.34 g, MgSO₄.7H₂O 0.05 g, glycerol 1.0 g, mixed phytosterol 0.2 g, Triton X-100 0.2 g

Transformation medium (100 ml): (NH₄)SO₄ 1.0 g, Na₂HPO₄ 0.45 g, KH₂PO₄ 0.34 g, MgSO₄.7H₂O 0.2 g, mixed phytosterol 2.0 g, mixture of Triton X-100 and Triton X-114 (1:1) 10.0 g

Microbial Cultivation

The seed culture was grown aerobically at 28° C. at 220 r/min for 3 days with 20 ml of medium in a 250 ml Erlenmeyer flask. The seed culture was then transferred by 10% into 22 ml of transformation medium in a 250 ml Erlenmeyer flask, which was then shaken at 28° C. at 220 r/min for 7 days. A portion of the well-mixed transformation culture broth was withdrawn for analysis.

Result

Total concentration of ADD and 4-AD (10:1) was 8.2 g/L, at a molar transformation rate of 76%.

### EXAMPLE 3

Microbial strain Mycobacterium sp. NRRL B 3683 is able to remove the side chain of cholesterol, giving out ADD and 4-AD as final products, at a ratio of ca. 10:1.

Media

Slant culture medium (100 ml): yeast extract 0.5 g, agar 1.2 g, glycerol 1.0 g, H₂KPO₄ 0.05 g, (NH₄)SO₄ 0.1 g, MgSO₄.7H₂O 0.05 g

Seed culture medium (100 ml): (NH₄)SO₄ 0.5 g, Na₂HPO₄ 0.45 g, KH₂PO₄ 0.34 g, MgSO₄.7H₂O 0.05 g, glycerol 1.0 g, cholesterol 0.2 g, Triton X-100 0.2 g

Transformation medium (100 ml): (NH₄)SO₄ 1.0 g, Na₂HPO₄ 0.45 g, KH₂PO₄ 0.34 g, MgSO₄.7H₂O 0.2 g, cholesterol 2.0 g, mixture of Triton X-100 and Triton X-114 (1:1) 10.0 g

Microbial Cultivation

The seed culture was grown aerobically at 28° C. at 220 r/min for 3 days with 20 ml of medium in a 250 ml Erlenmeyer flask. The seed culture was then transferred by 10% into 22 ml of transformation medium in a 250 ml Erlenmeyer flask, which was then shaken at 28° C. at 220 r/min for 7 days. A portion of the well-mixed transformation culture broth was withdrawn for analysis.

Result

Total concentration of ADD and 4-AD (10:1) was 6.45 g/L, at a molar transformation rate of 60%.

We claim:

1. A method to apply the cloud point system (CPS) in biotransformation, which is characterized in that one or more types of nonionic surfactant is selected to form a aqueous medium for microbial transformation whose cloud point is below the transformation temperature.

2. According to claim 1, a method to apply the CPS in biotransformation, which is characterized in that the nonionic surfactants thereof comprise polyoxyethyene alcohols, polyoxyethylene sorbitan fatty acid esters and alkylphenol ethoxylates.

3. According to claim 2, a method to apply the CPS in biotransformation, which is characterized in that the polyoxyethyene alcohols thereof comprise Brij 30, Brij 35, Brij 56 and C₁₂E₇; the polyoxyethylene sorbitan fatty acid esters thereof comprise Tween 20, Tween 40, Tween 60, Tween 80, Span 20, Span 40, Span 60 and Span 80; the alkylphenol ethoxylates thereof comprise Triton X-100 and Triton X-114.

4. According to claim 1, a method to apply the CPS in biotransformation, which is characterized in that the microbial transformation thereof is the microbial transformation where the substrate is hydrophobic, or microbial growth is inhibited by substrates or products, or product is further degraded by the microbes.
